(19) United States
(12) Patent Application Publication
(10) Pub. No.: US 2022/0133938 A1
(43) Pub. Date: May 5, 2022

(54) SYSTEM FOR HYGENIC GROUP EXERCISE

(71) Applicant: Lauren Brenner, Old Westbury, NY (US)

(72) Inventor: Lauren Brenner, Old Westbury, NY (US)

(21) Appl. No.: 17/169,909

(22) Filed: Feb. 8, 2021

Related U.S. Application Data
(60) Provisional application No. 63/108,407, filed on Nov. 1, 2020.

(51) Int. Cl. A61L 9/12; G16H 40/20; G05B 15/02; A63B 71/04; A61L 9/22; A61L 9/20; G01N 33/00

(52) U.S. Cl. CPC A61L 9/12; G16H 40/20; G05B 15/02; G16H 40/67; A61L 9/22; A61L 9/20; G01N 33/0063; A63B 71/04

(57) ABSTRACT

This invention relates to a combination of machinery, electronics and building space design using specialized disinfection systems, air filtration, audio-visual communication, electronic controllers and isolation booths in a manner that mitigates the risk of disease transmission among simultaneous and serial users of the shared space.

### PRIORITY CLAIM

This is a utility patent application which claims the benefit of U.S. Provisional Patent Application No. 63/108,407 filed on Nov. 1, 2020, which is hereby incorporated by reference in its entirety for all that it teaches herein.

### FIELD OF INVENTION

This invention relates to the design of an exercise facility that utilizes a combination of technologies in order to be sufficiently hygienic so as to control or eliminate transmission of disease between occupants using the facility, especially through exhaled airborne particles.

### BACKGROUND

Periodically throughout human history, epidemics sweep across society causing severe disruption to families and businesses. In some cases, fatality rates are high—causing misery among families. In other cases, hospitalization rates are high—causing economic stress on government institutions. In yet other cases, people may be permanently scarred or damaged by the disease. Even for relatively low death rate disease, the transmission rate may be very high. Influenza and corona viruses in general are highly transmissible from one person to a second as a result of the exhaling of the first person, and the inhaling of the second. This casual interaction can result in high transmission rates. As a result, people who are cognizant of the dynamics of epidemics of this sort avoid crowds, wear face masks and otherwise remain isolated. But this introduces another health risk: a sedentary lifestyle that leads to weight gain and heart disease. In normal times, people have to be encouraged to exercise, often by joining an exercise class that relies on group psychology to have a class leader motivate class members to exercise in a group. However, during epidemics, this becomes impossible without risking high rates of transfer of disease between the class members. For this reason, there is a need for a device or system that mitigates or eliminates the transmission of aerosol particles between class members while maintaining the interactivity between class members and a class leader in order that the class can meet its health goals through shared exercise.

### SUMMARY

This invention relates to a combination of machinery, electronics and building space design using specialized disinfection systems, air filtration, audio-visual communication, electronic controllers and isolation booths in a manner that mitigates the risk of disease transmission among simultaneous and serial users of the space.

### DESCRIPTION OF THE FIGURES

The headings provided herein are for convenience only and do not necessarily affect the scope or meaning of the claimed invention. In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced (e.g., element 204 is first introduced and discussed with respect to FIG. 2).

FIG. 1: an exemplary diagram of an isolation booth adapted for occupancy by a person.

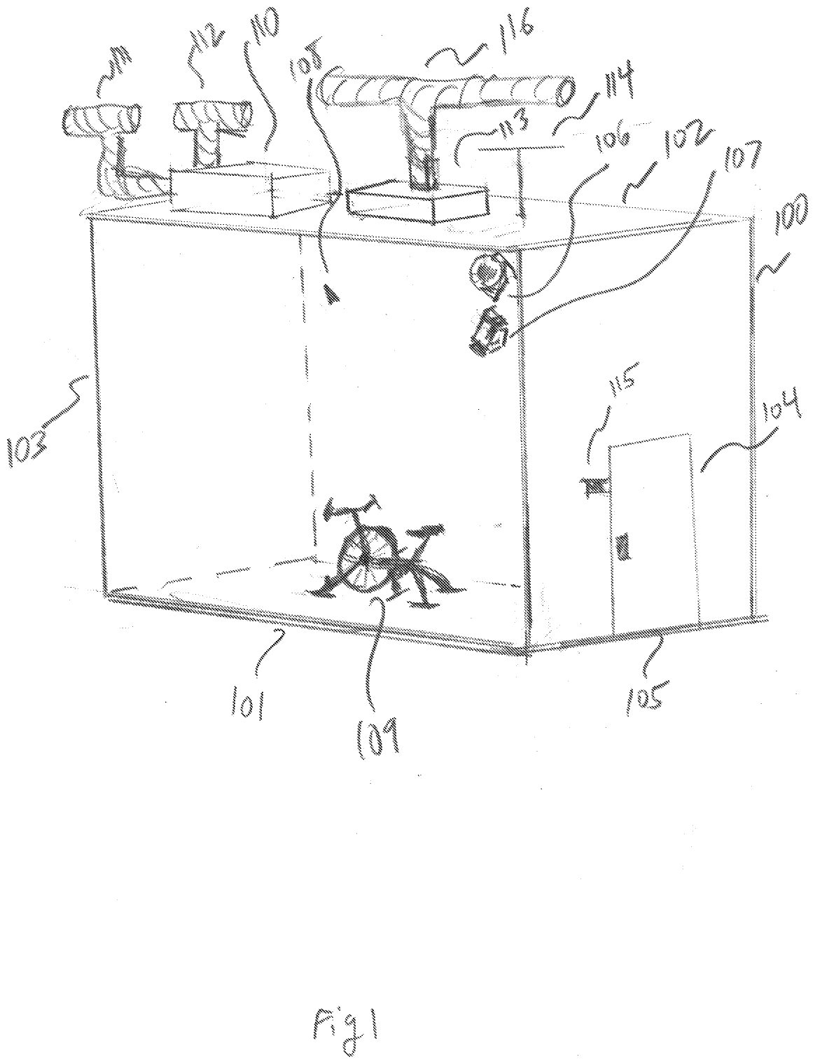

FIG. 2: an exemplary bottom view of the ceiling of the isolation booth.

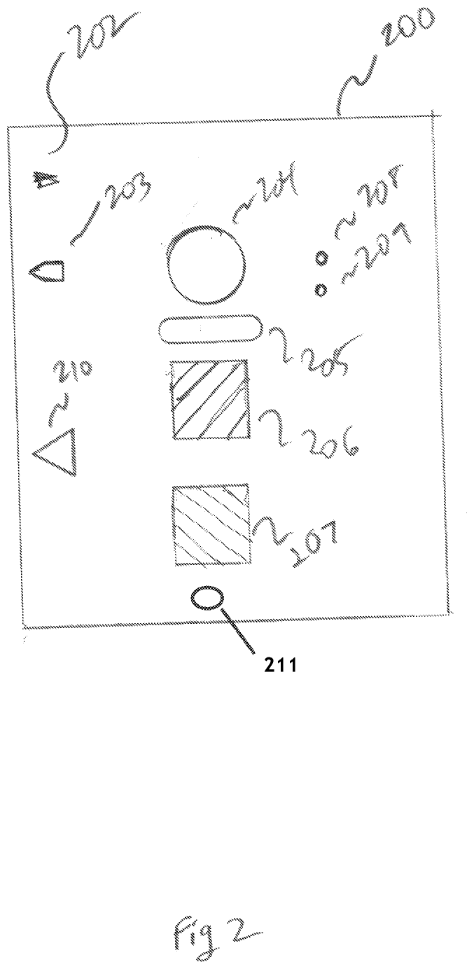

FIG. 3: an exemplary layout of multiple isolation booths comprising a facility.

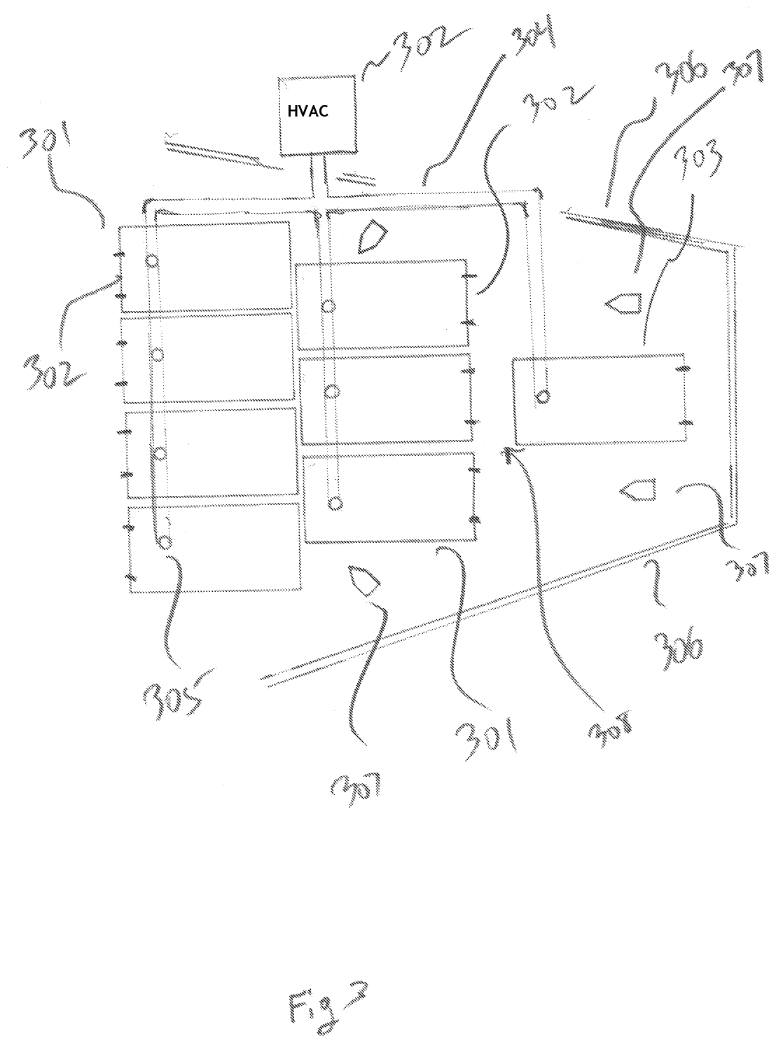

FIG. 4: an exemplary schematic of a portion of the audio-visual system.

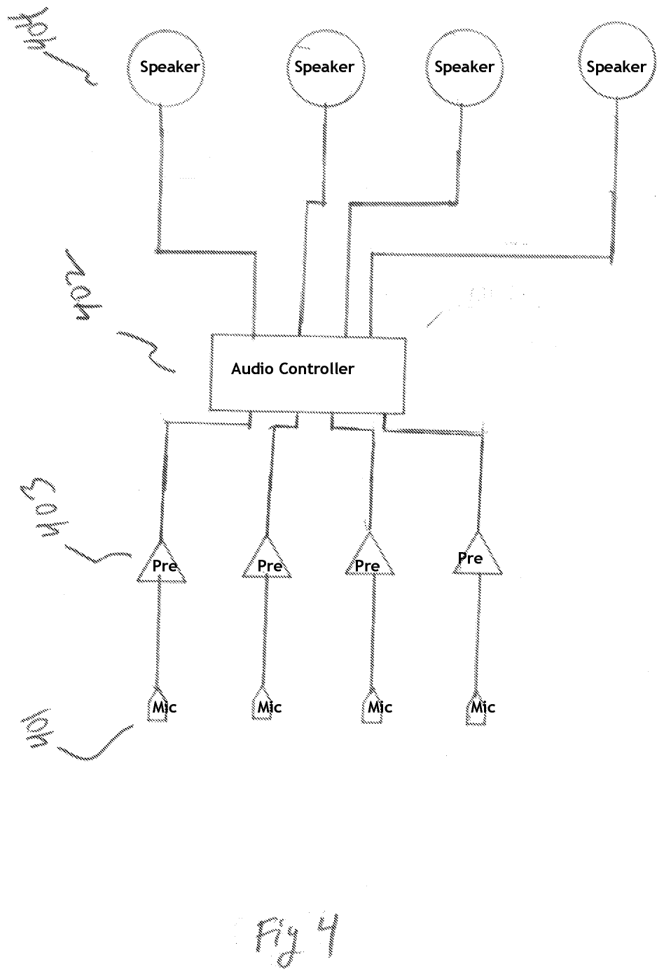

FIG. 5: an exemplary schematic of the electronic controllers comprising the system.

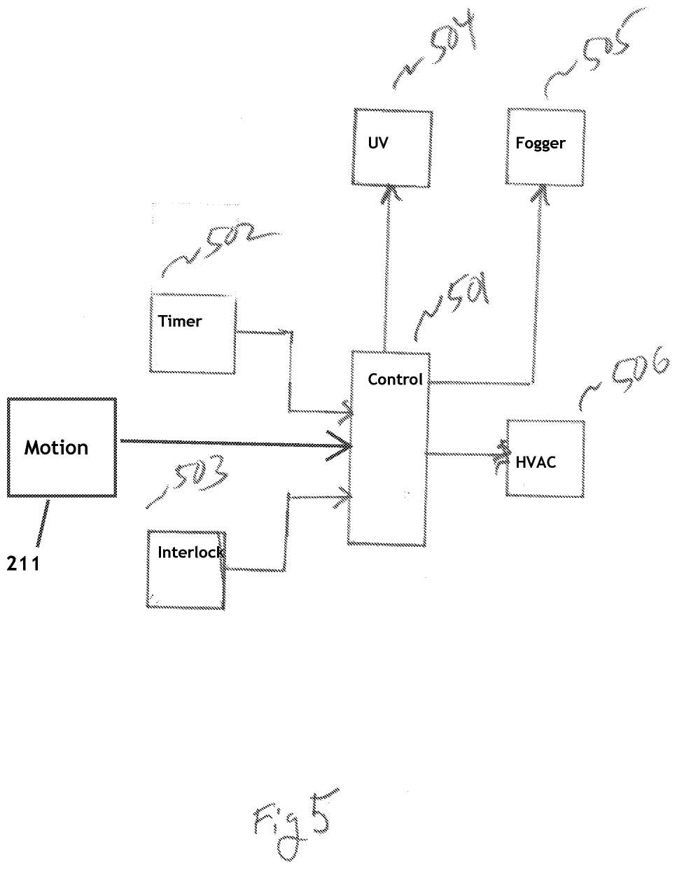

### DETAILED DESCRIPTION

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other features not described in detail herein. Additionally, some well-known structures or functions may not be shown or described in detail below, so as to avoid unnecessarily obscuring the relevant description. The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

The described embodiments of the invention are intended to be exemplary and numerous variations and modifications will be apparent to those skilled in the art. All such variations and modifications are intended to be within the scope of the present invention as defined in the appended claims. Although the present invention has been described and illustrated in detail, it is to be clearly understood that the same is by way of illustration and example only, and is not to be taken by way of limitation. It is appreciated that various features of the invention which are, for clarity, described in the context of separate embodiments may also be provided in combination in a single embodiment. Conversely, various features of the invention which are, for brevity, described in the context of a single embodiment may also be provided separately or in any suitable combination. It is appreciated that the particular embodiment described in the Appendices is intended only to provide an extremely detailed disclosure of the present invention and is not intended to be limiting.

The foregoing description discloses only exemplary embodiments of the invention. Modifications of the above disclosed apparatus and methods which fall within the scope of the invention will be readily apparent to those of ordinary skill in the art. Accordingly, while the present invention has been disclosed in connection with exemplary embodiments thereof, it should be understood that other embodiments may fall within the spirit and scope of the invention as defined by the following claims.

The invention is a system of interconnected isolation booths that together may be used to provide a participatory environment while maintaining hygienic isolation between participants. The booth (100) is comprised of a floor (101), a ceiling (102) and at least one wall (103). In one embodiment, the booth walls, floor and ceiling form a rhomboid. (100) In one embodiment, the floor dimensions are 9½ feet wide and 15 feet long. The ceiling is 10 feet high. In other embodiments, a curved surface may be used for the walls or ceiling. The isolation booth is designed and built to prevent air flow through the walls, at junctions of the walls with the floor, ceiling and other walls. The isolation booth is further comprised of a portal (104) that is also designed to prevent air flow along its edges (105) when it is closed. In one embodiment, the door is constructed as a rectangular shape. The wall of the booth has a rectangular opening with 4 edges. The wall and the door share at least two hinges that connect one edge of the door to one edge of the rectangular opening. Along the edges of the rectangular opening is a sealing strip, preferably made of rubber. The four edges of the rectangular opening, in one embodiment 3½ feet wide and 7 feet high, may have an additional edging strip that is recessed from the outer surface of the wall and supports the rubber sealing strip. The dimensions of the door is adapted so that when it closes, it settles into the rubber sealing strip against the recessed edging strip. In addition, the door is equipped with a sensor (115) that detects if the door has been opened. When the door (104) is opened, the sensor (115) transmits an alert data message to a controller (402) that includes the location or identity value corresponding to and thereby identifying the isolation booth.

The isolation booth further contains a loudspeaker (106), a video camera (107) and a microphone (108). The isolation booth may contain exercise equipment (109). However, in some cases the booth may have no equipment at all. In one embodiment, the exercise equipment (109) is a stationary cycle. In another embodiment, the exercise equipment is free weights, ropes, tires and body bars. In one embodiment the exercise equipment may be recessed within the floor.

The source of air in the isolation booth is through the HVAC system that services each booth (110). This device delivers air (111) that is filtered in order to remove microbes. For example, in one embodiment, the HVAC system may include a HEPA filter rated to remove infectious particles.

In one embodiment, the HVAC system delivers air through a filter with a MERV rating of 5 to 13. In another embodiment the HVAC system delivers air through a filter with rating 14 to 16. In other embodiments, the filter has a MERV rating of at least 13. In another embodiment, the HVAC system may include an electrostatic filter designed to trap particles smaller than 5 microns. In other embodiments, the electrostatic filter is designed to capture particles as small as 0.1 (one tenth) of a micron. The HVAC flow rate into each booth is adjusted so that the booth exhibits positive air pressure relative to the exterior of the booth. In this embodiment, the positive air pressure ensures that the air consumed within the booth was delivered through the HVAC system and not in through the door. The HVAC system also removes exhaust from the booth (112) but that waste airflow is exhausted from the facility. In one embodiment, the HVAC system is comprised of one air filtration or air purifier unit that is adapted to maintain airflow across it for a plurality of booths at the same time. In this embodiment, the airflow and air pressure in the supply duct downstream from air filter or air purifier has to be high enough to ensure there is no back-flow from one booth to another during operation. In another embodiment, each booth has its own air filtration or air purifier unit.

In one embodiment, an air content sensor is attached to the interior wall or ceiling of the booth. This device measures the relative concentration of Oxygen or Carbon Dioxide. The device is comprised of a microprocessor and associated circuitry that permits the device to communicate with the system controller over the data network. If the level of Carbon Dioxide rises above a predetermined threshold, or the Oxygen level is below a predetermined threshold, an alarm signal is generated and sent to the controller. Alternatively, the controller can routinely poll the sensors of all the booths in the facility and the computer program in the controller can execute program logic that determines if any of the sensors are detecting improper levels of Carbon Dioxide or Oxygen. In some embodiments, the air filtration or air purifier may be comprised of an Ultraviolet light source that illuminates the column of air passing through it at a sufficient intensity to act as a disinfectant.

The isolation booth further includes a nozzle or other delivery device (113) for distributing into the isolation booth a disinfecting fog. The disinfecting vapor or fog may be created electrostatically. In one embodiment, a reservoir for the disinfecting liquid is within the delivery device (113). In another embodiment, the reservoir is part of a central fog generating unit (302) that delivers fog vapor to the isolation booth (100) using a conduit (116). In one embodiment, the fog is a dry fog comprised of a hydrogen peroxide-based disinfectant. In another embodiment, the fog is a wet fog comprised of a chlorine dioxide or hydrogen peroxide-based solution.

The isolation booth (100) is connected by a computer network (114) to a centralized control station (501). The isolation booth is further comprised of an air sensor module (202) that detects the level of oxygen, or the level of carbon dioxide, or both, and transmits these levels and data across the network to the central controller module (501). The transmitting message from the sensor (202) is comprised of an index data value corresponding to the specific location and identity of the isolation booth that the sensor occupies. The controller module (501) is adapted to detect the condition that the effective oxygen level in an isolation booth is below a pre-determined safety threshold and to deliver an alert message to a pre-determined one or more destinations. The controller (501) is further comprised of a microprocessor and a computer memory with program code that when executed by the microprocessor, causes the controller module to receive the sensor module data and calculate whether or not a pre-determined safety threshold has been reached, form a data message comprising data representing a safety alert and to transmit the alert message to at least one a predetermined destination. The alert message is comprised of data indicating an identifier uniquely corresponding to the specific isolation booth that the low level of oxygen was detected in. In one embodiment, the alert message is transmitted as an audio signal to the loudspeaker (106) of a pre-determined isolation booth of a group of isolation booths in a facility (303). In another embodiment, the sensor (202) is further comprised of a microprocessor and a computer memory with program code that when executed by the microprocessors, causes the sensor module to determine whether or not a pre-determined saftey threshold has been reached and to transmit the alert to the central controller (501). In yet another embodiment, the alert message is comprised of audio that is routed through the audio controller module (402) to the loudspeaker comprising the identified isolation booth (100).

In one embodiment, the ceiling (200) of the isolation booth (100) is comprised of an HVAC outlet (207) that issues filtered air into the booth (100). In addition, the ceiling or walls may be comprised of an HVAC exhaust inlet (206) that removes exhaust air from the isolation booth (100). The ceiling (200) or wall may be comprised of a light for illumination (204). The ceiling or wall may be comprised of a video camera (203), a loudspeaker (208) and a microphone (209). The ceiling or wall may further be comprised of an outlet (210) for emitting disinfecting fog or vapor into the isolation booth (100). In addition, the ceiling or wall may be comprised of a ultra-violet light (205) for purposes of disinfecting the air and surfaces of the isolation booth (100) when it is not occupied. In other embodiments, there may be more than one ultraviolet light (205) located along the walls or the floor. In another embodiment, the ultraviolet light is controlled by a disinfecting controller (501). This local device contains logic for an interlocking mechanism between the door sensor (115) and the ultraviolet light (205). When the door sensor detects the condition of the door (104) opening, the disinfecting controller (501) turns off the ultraviolet light.

At least a portion of at least one of the walls (103) of the isolation booth (100) may be transparent. In one embodiment, the corners of the rhomboid (100) are structural material, for example struts of an opaque, load bearing nature like wood or steel. However, the walls themselves may be comprised of a transparent material supported by the vertical struts. In one embodiment, the transparent material is glass. In another embodiment, it is plexiglass or other transparent plastic. In yet another embodiment, the transparent material is comprised of an electrically responsive material that in reaction to an electric signal, selectively changes the transparent material into a translucent or opaque material. In this embodiment, the electrically responsive material comprising the wall is connected to a wall controller module. In one embodiment, the wall controller module receives input from a switch located in the isolation booth (100) such that the occupant can select whether the wall is transparent, translucent or opaque. In another embodiment, the wall controller is connected through the computer network (114) to the central controller module (501) in order that the central controller can also select the transparency of the wall.

The facility for use by a group of persons is comprised of at least two isolation booths that are proximate to each other (301). Each isolation booth has its own portal (302). In one embodiment the portal (302) is comprised of the transparent material described above. The at least one isolation booths that are proximate to each other (301) may be arranged with sufficient space between rows so as to form a hallway (308). However, the arrangement can be adapted to minimize the number of portals (302) that share the same hallway. In addition, there may be a predetermined isolation booth (303) that is at the front of the group of isolation booths. This predetermined isolation booth (303) may be used by the group leader. Because the walls (103) of all of the isolation booths are at least partially transparent, substantially all of the group participants utilizing the isolation booths (301) can see each other (305). The group of isolation booths (301) may be partially or wholly surrounded by a projection screen (306). The projection screen may display images or video projected from one or more display projectors (307). In another embodiment, the projection screen is comprised of an electronic display controlled by a computer or other electronic device without the use of a projector, for example, an LED screen. The group of isolation booths may be disinfected by a fog or vapor disinfectant delivered over dedicated conduit (304) from a central fog generating unit (302).

The sharing of sound and video is a further aspect of the invention. In this embodiment, each microphone (108; 401) in each isolation booth is routed to an audio control module (402, 403). The audio level from each microphone (401) may be adjusted using a pre-amplifier, (403), or an attenuation stage within a digital audio signal processing system comprising the audio control module (402). The audio controller (402) outputs an audio signal to the loudspeakers in the isolation booths (404, 106). In one embodiment, the audio control module (402) mixes the microphone signals (401) into one audio output that is distributed to all of the loudspeakers (404). In another embodiment, the audio control module (402) sets the relative loudness of the microphone (108) comprising the predetermined leader isolation booth (303) so that its signal is more prominent than the other microphones. In one embodiment, the loudspeakers (404) share the same audio output from the controller (402). In yet another embodiment, each loudspeaker (404) has a separate audio connection to the audio control module. Alternatively, the loudspeaker (108) may be comprised of a computer network connection such that the loudspeaker receives audio as a digital audio stream addressed to that loudspeaker. This permits selective audio to be delivered from the predetermined leader isolation booth (303) to a particular member of the participating group. In addition, each video camera (107) is connected to a video controller module. This may be accomplished by the local video camera converting the video to a data stream that is transmitted across the computer network (114). The video controller module can combine the incoming video feeds into one wall display, or receive a selection command to select one or more of the video camera data streams for producing a composite video output. The video output may be the projection screen. The video controller may be a component or module of the central controller (501). Alternatively, the predetermined leader isolation booth (303) may be further comprised of a video screen viewed by the instructing leader so that the leader can visually monitor what each participant is doing.

The central controller (501) may be comprised of either a microprocessor and a computer data memory comprised of program data or a logic circuit that when executed causes the central controller module to execute various control processes. In one embodiment, the central controller may be comprised of a timer module (502) and a door interlock sensor (503). In yet another embodiment, the isolation booth (100) may be comprised of a motion sensor (211) that is connected to the digital network (114) an thereby to the controller (501). The controller may be connected to each isolation booth's disinfecting system, for example the ultraviolet lights (504) and the disinfecting fog delivery system (505). In another embodiment, all of the ultraviolet lights and the entire fogging system (302) for a group of isolation booths are controlled together. In this embodiment, the controller program code or logic circuit (501) receives a command or signal to begin the disinfecting process for the group of isolation booths (301). In addition, the controller (501) may check or confirm by logic that the doors (104) of the isolation booths (100) are closed by means of the interlock sensor (115, 503). In addition, it may check the status of the motion detectors (211). If the program or electronic logic determines that the doors are closed and there is no motion in any of the isolation booths (301), the disinfection process can commence. This may reset a timer (502). The controller (501) can then cause the ultraviolet lights (504) to turn on and the fogging system (505, 302) to deliver the disinfecting fog or vapor through the conduit (304) to each isolation booth (116). When the timer module comprising the controller reaches a predetermined disinfection time, the controller (501) can cause the ultraviolet lights (504) to turn off and the disinfecting fog (505) to stop. At that point, the controller (501) can command the HVAC system controller (506) to cause the HVAC of each isolation booth (110) to exhaust the disinfecting fog through the conduit (112).

What is claimed:

1. A system for hygienic group exercise comprising:
a remote computer;
at least one exercise booths, each booth comprised of:
a door,
at least one wall; and
at least one electronic sensing device in data communication with the remote computer;
the system further comprising an air supply system that distributes into the at least one booth air under a predetermined pressure and a predetermined flow rate; and
at least one disinfectant delivery device that in operation, distributes into the at least one booth a disinfecting vapor or fog.

2. The system of claim 1 where in operation the disinfecting vapor or fog is created electrostatically.

3. The system of claim 2 where the vapor or fog is comprised of a hydrogen peroxide-based solution.

4. The system of claim 2 where the vapor or fog is comprised of a chlorine dioxide based solution.

5. The system of claim 1 where the air supply system is adapted so that when in operation it provides sufficient air pressure to maintain a positive air pressure within the at least one booths relative to the exterior of the at least one booth.

6. The system of claim 5 where the air supply system is adapted so that in operation maintains a positive pressure within each at least one booth sufficient to prevent during operation appreciable back-flow from a first at least one booth to a second at least one booth.

7. The system of claim 1 where the air supply system is comprised of at least one air purifier that is adapted to remove or destroy from the air passing through it into the at least one booth a predetermined percentage of infectious particles of a predetermined minimum average size.

8. The system of claim 7 where the air purifier is comprised of a filter with a MERV rating of about 5 to about 13.

9. The system of claim 7 where the air purifier is comprised of a filter with a MERV rating of at least about 13.

10. The system of claim 7 where the air purifier is comprised of a filter with a MERV ration of about 14 to about 16.

11. The system of claim 7 where the predetermined size is about five microns.

12. The system of claim 7 where the predetermined size is about one-tenth microns.

13. The system of claim 7 where the air purifier is an electrostatic air purifier.

14. The system of claim 1 where each booth is further comprised of a disinfecting radiation emitter mounted on at least one of a wall or a ceiling or a floor, that in operation illuminates at least a portion of the surfaces in the booth to a disinfecting radiation.

15. The system of claim 14 where the emitter in operation is controlled by an electronic circuit that in operation receives control data from the remote computer, where the receipt of the control data can cause the emitter to turn on or off.

16. The system of claim 15 where the disinfecting radiation is ultraviolet light.

17. The system of claim 1 where the electronic sensing device is an air quality sensor that in operation monitors the air quality within the respective booth and transmits air quality data to the remote control computer.

18. The system of claim 17 where the air quality data represents either the effective oxygen level or the condition that the effective oxygen level is below a pre-determined safety threshold.

19. The system of claim 1 where each booth is further comprised of a loudspeaker system that in operation receives audio data from the remote control computer and renders the received audio data into sound waves.

20. The system of claim 1 where the electronic sensing device is a video camera system and a microphone system that in operation transmits data representing audio and video to the remote control computer

21. The system of claim 1 where the electronic sensing device is a motion sensor that in operation transmits motion data the remote control computer.

22. The system of claim 1 where each booth is further comprised of an interlock switch that in operation detects the condition of the door being opened, said interlock switch adapted to transmit to the remote computer data representing either an open state or closed state of the door.

23. The system of claim 22 where the remote computer is comprised of a computer data memory comprised of program code that when executed causes the remote computer to:
receive from the interlock of one of the at least one booths a data message representing the condition that the door is open; and
receive data representing the location or identity of the one booth with the open door.

24. The system of claim 23 where each at least one booth is further comprised of a disinfecting radiation emitter and the computer data memory is further comprised of program code that when executed causes the remote computer to transmit a command to turn off the emitter comprising the booth corresponding to the location or identity of the booth with the open door.

25. The system of claim 17 where the remote computer is comprised of a computer data memory comprised of program code that when executed causes the remote computer to:
receive from the air quality sensor of one of the at least one booths a data message representing the condition that the air quality is below a predetermined threshold; and
receive data representing the location or identity of the one booth with the air quality below the predetermined threshold.

26. The system of claim 25 where the computer data memory is further comprised of program code that when executed causes the remote computer to transmit data representing an alert to the booth corresponding to the location or identity of the booth with the air quality below the predetermined threshold.

27. The system of claim 1 further comprising:
at least one electric wall controller circuit,
where the at least one wall in each booth is comprised of a material that can be selectably switched between a first state where the wall is substantially transparent and a second state where the wall is substantially opaque by using the at least one electric wall controller circuit.

28. The system of claim 1 where each booth is adapted to prevent air flow through the wall, at junctions of the walls with a floor, a ceiling and another wall.

29. The system of claim 1 where the door comprising each booth is adapted to prevent air flow along its edges when it is in a closed position.

30. The system of claim 29 where each at least one booth is comprised of an opening that receives the door and is further comprised of a sealing strip and the dimensions of the door is adapted so that when the door is in the closed state, it has settled into the sealing strip.

31. The system of claim 1 where a display screen positioned along at least a portion of a locus surrounding the at least one booths and oriented so that in operation the usable side of the display is at least partially visible from the at least one booths.

32. The system of claim 31 where the display screen is comprised of an LED screen.

33. The system of claim 31 further comprising at least one projector, the at least one projectors oriented so that in operation, the projectors project images or video onto the display screen.

34. The system of claim 22 where the remote computer is comprised of a computer data memory comprised of program code that when executed causes the remote computer to:
confirm by program logic taking as input the data received from the corresponding at least one interlock sensors that the doors of the at least one booths are in the closed state;
confirm by program logic taking as input the data received from the corresponding at least one electronic sensing devices representing the condition that there is no motion inside the at least one booths; and
in dependence on both confirmations, transmit commands to the disinfection delivery device to activate delivery of the disinfecting vapor or fog into the at least one booths.

35. The system of claim 34 where the computer memory is further comprised of program code that when executed causes the system to:
Determine the condition that a predetermined time has elapsed since the disinfection delivery device was activated;
In dependence on the determination, stop the delivery of the disinfecting fog or vapor.

36. The system of claim 35 where the system is further comprised of an air evacuation system and the computer memory is further comprised of program code that when executed causes the system to exhaust the disinfecting fog out through the air evacuation system.
